(19) United States
(12) Patent Application Publication
(10) Pub. No.: US 2026/0092802 A1
(43) Pub. Date: Apr. 2, 2026

(54) TDR-BASED DETECTION OF CONTAMINATION

(71) Applicant: Endress+Hauser SE+Co. KG, Maulburg (DE)

(72) Inventors: Johannes Baureithel, Kami-Imai-cho (JP); Stefan Scherr, Kandern (DE); Andreas Frischknecht, Fuefuki-shi (JP); Ralf Reimelt, Freiburg (DE); Keita Okazaki, Hokuto-shi Yamanashi (JP); Thomas Blödt, Steinen (DE); Kouta Hirose, Yamanashi (JP)

(21) Appl. No.: 19/347,154

(22) Filed: Oct. 1, 2025

(30) Foreign Application Priority Data
Oct. 2, 2024 (DE) 10 2024 128 487.0

(51) Int. Cl. G01F 23/284; G01F 23/76; G01F 23/80; G01N 33/18

(52) U.S. Cl. CPC G01F 23/284; G01F 23/76; G01F 23/802; G01N 33/1833

(57) ABSTRACT

A measuring system for TDR-based detecting of contamination on a surface of a liquid medium comprises: a measuring probe having an end region extending into the medium; a TDR unit designed to couple a signal into the measuring probe and, after passage through the measuring probe, to out-couple a corresponding received signal; and an evaluation unit to digitize the received signal by undersampling and, based on the digitized received signal, to detect, whether any wetting of the measuring probe with the medium is present, and whether a contamination is present on the surface of the measured medium. Advantageous in the measuring system of the present disclosure is a compact and low power adaptability, whereby the measuring system can be arranged on a compact buoyancy body for the measured medium.

### CROSS-REFERENCE TO RELATED APPLICATION

The present application is related to and claims the priority benefit of German patent application no. DE 10 2024 128 487.0, filed on Oct. 2, 2024, the entire contents of which are incorporated herein by reference.

### TECHNICAL FIELD

The present disclosure relates to a TDR-based measuring system for detecting of contamination in catch basins.

### BACKGROUND

In certain process installations, catch basins are provided as barriers in order, for example, in case of damage, to prevent or at least to delay contamination of the environment with poisonous substances. Thus, industrial oil storage tanks often contain an oil retention basin as catch basin. Industrial large pumps are, as a rule, designed with a pump sump as catch basin. When a measuring system for detecting a potential contamination is installed on such catch basins, corresponding steps for leakage-fighting on the particular process installation can be initiated based on the results of measurement.

### SUMMARY

A suitable measuring system for this is manufactured and sold by the Endress+Hauser group of firms under the product designation “NAR300”. Such a measuring system includes a buoyancy body, which floats in the catch basin and on which a vibronic sensor and a conductivity sensor are arranged. In such case, the vibronic sensor serves for checking whether the buoyancy body is floating or lies dry due to an empty catch basin. Based on the conductivity measured value, information can be obtained concerning whether the water is contaminated by a contamination, such as an oil layer or a least concentration of, for example, ethanol or methanol. Because such a measuring system must, however, comprise two different sensors, or sensor types, it is difficult, on the one hand, to meet certain explosion protection specifications. On the other hand, the underpinning buoyancy body is to be constructed correspondingly voluminously. This makes difficult the use of such measuring system in smaller catch basins and in process installations with high explosion protection requirements. An object of the present disclosure, therefore, is to provide a measuring system for detecting contamination in catch basins and overcoming these disadvantages.

The present disclosure achieves the object by a measuring system for TDR-based detecting of contamination on a surface of a liquid, measured medium, comprising: at least one measuring probe, which is arranged in such a manner that at least one probe end region extends into the measured medium; a TDR unit, which is designed according to the TDR principle to couple a signal into the measuring probe, and after passage through the measuring probe, to out-couple a corresponding received signal; and an evaluation unit, which is designed to digitize the received signal especially by means of undersampling, and, based on the digitized received signal, to detect whether a wetting of the measuring probe with the measured medium is present, and whether a contamination is present on the surface of the measured medium when the measuring probe is wetted with measured medium.

According to the present disclosure, the function of vibronic sensor and the function of conductivity sensor can be replaced by using the TDR principle. When the measuring system of the present disclosure is, thus, based on a buoyancy body for the measured medium and at least the measuring probe is arranged on the buoyancy body, the buoyancy body can be correspondingly compactly designed. In such case, it is within the scope of the present disclosure likewise an option not to arrange the measuring probe on a buoyancy body, but, instead, stationarily on a container wall. Advantageous in the design of the present disclosure is, moreover, that the measuring system, thus the TDR unit and the evaluation unit, are easily designable to be explosion protection conforming, since two sensors do not need to be supplied with power.

In general, the TDR principle (“Time Domain Reflectometry”) rests on impressing a pulse shaped voltage signal clocked into the electrically conductive measuring probe, wherein the measuring probe is in contact with the investigated measured medium. The pulse shaped signal is reflected in the measuring probe, wherein the signal amplitude of the reflected pulses depends on the conductivity of the measured medium surrounding the measuring probe. Thus, with evaluation of the reflected received signal—in the simplest case by evaluating the amplitude—the properties of the measured medium can be determined. Due to the fast signal travel time of the clocked pulses, a determining of the amplitude is circuit-wise, however, quite complex. Therefore, the received signal is correspondingly time expanded and discretized by undersampling. In connection with fill level measurement, the TDR principle is described, for example, in US patent 10,07,743 B2.

Depending on design, the evaluation unit can detect a possible contamination, for example, by means of a classic or a machine learnable, classification algorithm. Implemented as machine learnable classification algorithm in the evaluation unit for this can be, for example, an especially end-to-end learning based, deep learning method. Alternatively, a main components analysis can be implemented in the evaluation unit, in order to detect a possible contamination.

The classification algorithm is adapted to the particular type of measured medium, thus, for example, water. Accordingly, the evaluation unit is to be constructed to detect as contamination, for example, an oil layer, an ammonia least concentration, an alcohol least concentration or a bicarbonate least concentration in the measured medium, especially in water. In such case, of concern can also be salt containing water, e.g., ocean water. The terminology alcohol includes in this connection both ethanol as well as also methanol.

The form design of the measuring probe is, in principle, not fixedly predetermined. In the simplest case, the measuring probe can be rod shaped. Another option is, however, a spiral shape, in order to lengthen the path length of the signal and, as a result, increase the potential measuring-resolution. Essential in this connection is only the electrical conductivity of the measuring probe. Provided as ground electrode can be, for example, the buoyancy body, when such is made of an electrically conductive material. However, also a separate ground electrode can be provided, which has, for example, a shape identical to the measuring electrode.

Understood under the concept unit in the context of the present disclosure is, in principle, any electronic circuit suitably designed for the intended application. It can, thus, depending on requirements, be an analog circuit for producing and processing corresponding analog signals. It can, however, also be a digital circuit such as an FPGA or a storage medium in cooperation with a program. In such case, the program is designed to perform the corresponding method steps, or to apply the needed computer operations of the unit. In this context, different electronic units of the measuring system can in the sense of present disclosure potentially also use a shared physical memory, or be operated physically by means of the same digital circuit.

### BRIEF DESCRIPTION OF THE DRAWINGS

The present disclosure will now be explained in greater detail based on the appended drawing, the sole FIGURE of which show as follows:

FIG. 1 shows a measuring system of the present disclosure on a container.

### DETAILED DESCRIPTION

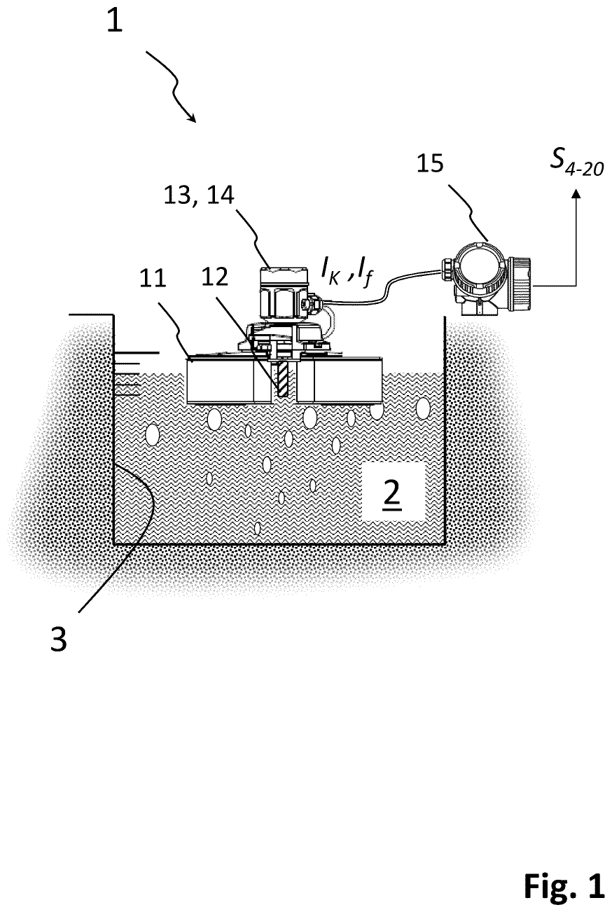

For providing a basic understanding of the present disclosure, FIG. 1 shows a catch basin 3, which functions, for example, for collecting oil residues of an oil storage tank. Catch basin 3 is filled with water 2, wherein the water level can, in given cases, change due to various factors, such as precipitation. In the case of a leakage from the associated oil storage tank, an oil layer forms on the water surface. In order to detect such a case of disturbance, there floats on the water surface a measuring system 1 of the present disclosure, wherein the measuring system 1 comprises for this a corresponding buoyancy body 11.

For detecting a possible oil layer or other contamination, the measuring system 1 is based according to the present disclosure on the TDR principle. Accordingly, secured on an upper side of the buoyancy body 11 are a TDR unit 13 and an evaluation unit 14. Extending from the TDR unit 13 and from the evaluation unit 14 is a measuring probe 12, in such a manner rod shaped and downwards that the measuring probe 12 in the floating state penetrates, at least partially, the water surface and accordingly the lower probe end region penetrates into the water 2. A corresponding hollow space is formed for the measuring probe 12 within the buoyancy body 11. As can be seen from FIG. 1, the measuring probe 12 does not extend beyond a floor-plane of the buoyancy body 11. In this way, it is assured that, in the case of emptying of the catch basin 3, the buoyancy body 11 rests stably on the container floor. When the buoyancy body 1 is made of metal, it can function as ground electrode for the measuring probe 12.

The TDR unit 13 couples according to the TDR principle an electrical signal of pulse shape into the measuring probe 12. In such case, for implementing the TDR principle, the TDR unit 13 can operate, for example, based on a capacitor, which is discharged for producing the pulse lasting 100 ps up to about 1 ns. In order that the signal is produced according to the TDR method with the required pulse rate between 100 kHz and 1 MHz, the capacitor can be operated correspondingly clocked within the TDR unit 13. The TDR unit 13 can couple the signal into and out of the measuring probe 12, for example, via a transmitting/receiving separator (not shown). In such case, the design of the transmitting/receiving separator is, in principle, not fixedly predetermined. It can, for example, be designed purely as electrical nodes.

In the measuring probe 12, the pulse shaped signal is partially reflected by the jump of the dielectric constant for the water 2. Additionally, another essential part of the signal is reflected at the probe end region opposite the signal input/output of the measuring probe 12 for in- and out-coupling of the signal. Accordingly, the reflected, pulse shaped signals are correspondingly received by the evaluation unit 14 of the measuring device 1 as received signal according to corresponding signal travel times through the measuring probe 12. For this, the evaluation unit 14 is, in turn, connected to the measuring probe 12 via the transmitting/receiving separator. In such case, the strength, or amplitude, of the received signal depends decisively on the dielectric constant and the conductivity σ of the water 2.

Due to the fast signal travel time of the signal pulses, it is difficult without suitable technical measures metrologically to register the characteristics of the received signal, such as the signal travel time and the signal strength. Therefore, the evaluation unit 14 is designed to expand the reflected, received signal in signal direction behind the transmitting/receiving separator by undersampling in the time axis. For this, the evaluation unit 14 can preferably comprise a digital sampler, which mixes the received signal with electrical sampling pulses.

The sampling rate, with which the sampling pulses are produced, differs by a defined factor of greatly less than 0.1 promille from the clocking rate of the produced and transferred signal pulses. For producing the sampling pulses, the evaluation unit 14 can, in turn, comprise a capacitor and a corresponding reference clock signal generator. Advantageous with the time expansion is that the received signal after undersampling compared with the pure received signal is technically significantly simpler to process.

By undersampling, the received signal is, moreover, digitized. This favors according to the present disclosure evaluation of the received signal as regards a possible contamination of the water 2: In the evaluation unit 14 for this, for example, a main components-analysis or an especially machine learnable classification algorithm, such as an end-to-end learning based, deep learning method can be implemented. This enables that the evaluation unit 14 learns to detect in a learning phase the contamination for the concrete case of application based on corresponding received signals. In such case, for example, an oil layer on the water 2 or an alcohol least content in water 2 can be trained as contamination. In the case of offshore installations, the measured medium can also concern salt containing water 2.

Besides the detecting of a possible contamination, the evaluation unit 14 is, moreover, capable, based on the received signal, of finding out, whether the measuring electrode 12 is at least partly wetted, or not, with—in given cases, contaminated—water 2. From such, the measuring system 1 can, in turn, infer, whether the buoyancy body 11 lies dry on the floor of the catch basin 3 due to water shortage.

Accordingly, the measuring system 1 of the present disclosure, thus the evaluation unit 14, is able to output at least two pieces I_{K}, I_{f} of information:
I_{f}: Does the buoyancy body lie dry (yes/no)?
I_{K}: Is a contamination present (yes/no)?

When a contamination is present, then, depending on type of contamination and depending on evaluation of the received signal, in given cases, supplementally the degree of the contamination can be output, for example, in the form of a layer thickness or in the form of a concentration.

In the embodiment shown in FIG. 1, the measuring system 1 of the present disclosure includes a separate transmitter unit 15, via which at least these two pieces I_{f}, I_{K} of information can be output to a superordinated process control station. Transmitter unit 15 is, in such case, secured at a fixed location outside of the catch basin 3. In such case, the transmitter unit 15 converts the information I_{f}, I_{K} produced by the evaluation unit 14 into that protocol, such as, for example, 4-20 mA, for which the process installation is designed. Besides a cable-bound protocol, in principle, also a wireless communication between transmitter unit 15 and the process control is an option, such as WLAN or LTE.

In FIG. 1, the TDR unit 13 and the evaluation unit 14 are connected by cable with the transmitter unit 15, in order, on the one hand, to transmit the information I_{f}, I_{K} registered by the evaluation unit 14 relative to the possible contamination and the wetting of the measuring probe 12 with water, or measured medium 2. On the other hand, the TDR unit 13 and the evaluation unit 14 can be supplied with electrical current by means of this cable connection. In such case, the construction of the present disclosure for the measuring system 1 facilitates an explosion protection conforming, electrical current saving design, since in contrast to the state of the art two different sensors do not need to be operated, in order to be able to determine the two pieces I_{f}, I_{K} of information. Advantageous in this connection is, moreover, that, the buoyancy body 11 can be designed with correspondingly small buoyancy and correspondingly compactly.

In contrast with the embodiment shown in FIG. 1, the measuring system 1 of the present disclosure does not, however, have to have a buoyancy body 11. Alternatively, the measuring probe 12, the TDR unit 13 as well as the evaluation unit 14 can be secured to an inner surface of the catch basin 3. In such case, whether the measuring probe 12 is wetted with measured medium 2 or with water and whether a minimum water level is subceeded in the catch basin 3 is ascertained and output as basis for information I_{f}.

1. A measuring system for a Time Domain Reflectometry (TDR)-based detecting of a contamination on a surface of a liquid, measured medium, the measuring system comprising:
a measuring probe arranged such that a probe end region extends into the measured medium;
a TDR unit designed according to the TDR principle to couple a signal into the measuring probe, and, after passage of the signal through the measuring probe, to out-couple a corresponding received signal; and
an evaluation unit designed to digitize the received signal by undersampling, and, based on the digitized received signal, to detect whether a wetting the measuring probe with the measured medium is present and whether the contamination is present on the surface of the measured medium when the measuring probe is wetted with measured medium.

2. The measuring system as claimed in claim 1, further comprising:
a buoyancy body for the liquid, measured medium,
wherein the measuring probe is arranged on the buoyancy body.

3. The measuring system as claimed in claim 1, wherein the evaluation unit is designed to detect the contamination by a main components analysis or by a machine-learnable classification algorithm.

4. The measuring system as claimed in claim 3, wherein an end-to-end learning based, deep-learning method is implemented as the machine-learnable classification algorithm.

5. The measuring system as claimed in claim 4,
wherein the liquid, measured medium is water, and
wherein the evaluation unit is designed to detect as the contamination at least one of the following: an oil layer, an ammonia least concentration, an alcohol least concentration, and a bicarbonate least concentration in the liquid, measured medium.

6. The measuring system as claimed in claim 1, wherein the TDR unit and the evaluation unit are designed to be explosion protection conforming.

7. The measuring system as claimed in claim 1, wherein the measuring probe is rod-shaped.

8. The measuring system as claimed in claim 1, wherein the measuring probe is spiral-shaped.

9. The measuring system as claimed in claim 2,
wherein the TDR unit and the evaluation unit are secured on an upper side of the buoyancy body,
wherein the buoyancy body is embodied with a hollow space on a lower side of the buoyancy body, and
wherein the measuring probe is disposed in the hollow space on the lower side of the buoyancy body and extends through the hollow space only to a floor plane of the buoyancy body.
